(19) United States
(12) Patent Application Publication
(10) Pub. No.: US 2004/0002168 A1
(43) Pub. Date: Jan. 1, 2004

(54) Apparatus and methods for detecting cerebrospinal fluid

(75) Inventors: Benjamin J. Remington, Modesto, CA (US); David J. Bearss, Modesto, CA (US); Kavian Shahi, Granite Bay, CA (US)

Correspondence Address: WILSON SONSINI GOODRICH & ROSATI, 650 PAGE MILL ROAD, PALO ALTO, CA, 943041050

(73) Assignee: NeuroPro Technologies, Inc., Salida, CA (US)

(21) Appl. No.: 10/460,742

(22) Filed: Jun. 11, 2003

Related U.S. Application Data
(60) Provisional application No. 60/388,537, filed on Jun. 13, 2002.
(60) Provisional application No. 60/394,806, filed on Jul. 10, 2002.

(51) Int. Cl. G01N 33/543; C12M 1/34; C07K 16/18; C07K 16/46

(52) U.S. Cl. 436/518; 435/287.2; 530/388.25

(57) ABSTRACT

Antibodies, apparatus and methods of detecting and diagnosing cerebrospinal fluid and conditions associated therewith are disclosed. In particular embodiments, CSF specific proteins are detected with antibodies in a strip test.

### REFERENCES TO PRIOR APPLICATIONS

This application claims priority to Provisional Patent Application No. 60/388,537, filed Jun. 13, 2002 and No. 60/394,806, filed Jul. 10, 2002, entitled “Apparatus and Method For Detecting Cerebrospinal Fluid.” The above applications are hereby incorporated by reference in their entirety.

### BACKGROUND

Each year in the United States over 1 million people are treated and released from hospital emergency departments with traumatic head injuries. An additional 230,000 people are hospitalized with traumatic head injuries each year. Approximately 2-3% of head injuries result in cerebrospinal fluid leakage. Cerebrospinal fluid leaks are, therefore, a relatively common condition seen in hospital emergency rooms and are becoming one of the more persistent problems encountered in neurosurgical practice.

Cerebrospinal fluid leakage can lead to rhinorrhea and otorrhea, which commonly result from trauma, either accidental or iatrogenic (surgery, etc.). Nontraumatic causes for cerebrospinal fluid leakage include elevated intracranial pressure, congenital abnormalities, and osteomyelitic erosion. In rare cases, cerebrospinal fluid rhinorrhea may occur spontaneously or independently of trauma. While cerebrospinal fluid leakage may be traumatic, iatrogenic, or spontaneous in origin, it affects a relatively large proportion of neurosurgical patients. With traumatic cases, the onset of leakage may be delayed by months or years as blood clots are reabsorbed or tissues slowly remodeled.

If cerebrospinal fluid leakages are not repaired, they may lead to various ailment conditions including but not limited to meningitis, cerebritis, brain abscess, chronic headaches, neck aches or hearing loss. As diagnosis of cerebrospinal fluid leakage is difficult, inaccurate and time consuming, patients may require hospitalization until results from laboratory tests are obtained and confirmed. A faster, more accurate detection system for cerebrospinal fluid leakage is desirable and disclosed herein.

### SUMMARY OF THE INVENTION

The present invention discloses methods, kits and reagents that can be directly used to detect cerebrospinal fluid leakage. In one specific embodiment, the present invention provides monoclonal antibodies that specifically bind beta-2 transferrin and apparatus and methods for detecting cerebrospinal fluid leakage.

### SUMMARY OF THE FIGURES

FIG. 1 illustrates binding of commercial transferrin antibodies.

FIG. 2 illustrates binding of beta-2 transferrin antibodies herein.

FIG. 3 illustrates binding of commercial transferrin antibodies and beta-2 transferrin antibodies.

FIG. 4 illustrates an embodiment of a detection apparatus.

FIG. 5 illustrates an embodiment of a detection apparatus.

FIG. 6 illustrates an embodiment of a detection apparatus.

FIG. 7 illustrates a method for detection of cerebrospinal fluid leakage herein.

FIG. 8 illustrates clinical results from clinical tests of four unknown samples.

FIGS. 9A-D illustrate potential assay results.

FIG. 10 illustrates the amino acid sequence of human transferrin.

### DETAILED DESCRIPTION OF THE INVENTION

A. Terminology

To facilitate an understanding of the present invention, a number of terms and phrases are defined below:

As used herein, the term “adjuvant” is defined as a substance which enhances the immunogenicity of a coadministered antigen. It is not intended that the present invention be limited to any particular type of adjuvant or that the same adjuvant, once used, be used in all subsequent immunizations. Examples of adjuvants, include but are not limited to, keyhole limpet hemocyanin (KLH), agar beads, aluminum hydroxide or phosphate (alum), Freund's adjuvant (incomplete or complete), Quil A adjuvant and Gerbu adjuvant (Accurate Chemical and Scientific Corporation), and bacterins (i.e., killed preparations of bacterial cells, especially mycoplasma).

As used herein, the terms “antibody” and “antibodies” refer to any immunoglobulin that binds specifically to an antigenic determinant. Examples of antibodies include, but are not limited to, monoclonal antibodies, polyclonal antibodies, humanized antibodies, chimeric antibodies, single chain antibodies, Fab fragments, F(ab′)₂ fragments, fragments produced by FAb expression library, anti-idiotypic (anti-Id) antibodies, epitope-binding fragments of any of the above. Antibodies can be any immunoglobulin (e.g., IgG, IgM, IgA, IgE, IgD, etc.) obtained from any source (e.g., humans, rodents, non-human primates, lagomorphs, caprines, bovines, equines, ovines, etc.). In some embodiments, an antibody is directed against a species (e.g., anti-mouse, anti-human, etc.).

As used herein, the term “antigen” refers to any substance that is capable of generating an immune response (e.g., the production of antibodies).

As used herein, the term “antigenic determinant” or “epitope” refers to that portion of an antigen that makes contact with a particular antibody variable region.

As used herein, the term “anti-species antibody” refers to an antibody that is made by immunizing one species of animal with the antibody from another species. For example, if a primary antibody is made with mouse antibodies, then a rabbit immunized mouse antibodies will produce rabbit anti-mouse antibodies.

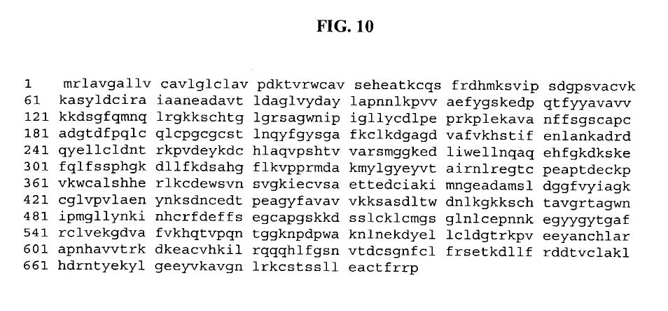

As used herein, the terms “transferrin” refer collectively to all isoforms of a protein having an amino acid sequence illustrated in FIG. 10.

As used herein, the term “beta-i transferrin” refers to one or more isoforms of transferrin wherein the isoform has one or more sialic acid side residues.

As used herein, the terms “beta-2 transferrin” refer to one or more isoforms of human transferrin having no sialic acid side residues.

As used herein, the term “capture antibody” refers to an antibody that is immobilized onto a substrate and serves to capture an antigen or an antigen-antibody complex.

As used herein, the term “CSF-specific protein” refers to any protein that is located in the cerebrospinal fluid (CSF), perilymph, humor and/or other bodily fluids in association with CSF.

As used herein, the terms “detection antibody” and “detector antibody” refer to an antibody, which carries a means for visualization or quantification. Preferably a detection antibody is conjugated to colloidal gold or other detectable reagent for visualization and/or quantification. In alternative embodiments, such antibody may be conjugated to an enzyme moiety that typically yields a colored or fluorescent reaction product following the addition of a suitable substrate. Commonly used conjugated enzymes include horseradish peroxidase, urease, alkaline phosphatase, glucoamylase and beta-galactosidase. In some embodiments, the detection antibody is directed against an antigen of interest (e.g., human beta-2 transferrin), while in other embodiments, the detection antibody is not directed against the antigen of interest but is an anti-species antibody. In further alternative embodiments, the detection antibody can be prepared with a label such as biotin, a fluorescent marker, or a radioisotope, and is detected and/or quantified using this label.

As used herein, the term “immunoassay” refers to any assay that uses at least one specific antibody for the detection and/or quantification of an antigen. Immunoassays include, but are not limited to, rapid strip tests, Western blots, ELISAs, radio-immunoassays, and immunofluorescence assays and any other antigen-antibody reactions including, for example, “flocculation” (i.e., a colloidal suspension produced upon the formation of antigen-antibody complexes), “agglutination” (i.e., clumping of cells or other substances upon exposure to antibody), “particle agglutination” (i.e., clumping of particles coated with antigen in the presence of antibody or the clumping of particles coated with antibody in the presence of antigen), “complement fixation” (i.e., the use of complement in an antibody-antigen reaction method), and other methods commonly used in serology, immunology, immunocytochemistry, immunohistochemistry, and related fields.

As used herein, the terms “detectable reagent,” “label” and “reporter” refer to any substance which can be attached to specific binding agents, such as antibodies or antigens, which is capable of producing a signal that is detectable by visual or instrumental means. Various suitable labels for use in the present invention can include chromogens, catalysts, fluorescent compounds (such as, for example, fluorescein, phycobiliprotein, rhodamine), chemiluminescent compounds, radioactive elements, colloidal metallic (such as gold), non-metallic (such as selenium) and dye particles, enzymes, enzyme substrates, and organic polymer latex particles, liposomes or other vesicles containing such signal producing substances, and the like. Examples of enzymes that can be used as labels include phosphatases and peroxidases, such as alkaline phosphatase and horseradish peroxidase which are used in conjunction with enzyme substrates, such as nitro blue tetrazolium, 3,5′,5,5′-tetranitrobenzidine, 4-methoxy-1-naphthol, 4-chloro-1-naphthol, 5-bromo-4-chloro-3-indolyl phosphate, chemiluminescent enzyme substrates such as the dioxetanes.

As used herein, the term “kit” refers to a combination of reagents and/or apparatus, which facilitates sample analysis. In some embodiments, a kit may further include one or more antibodies, reporter reagents, antigens, epitopes, and/or written instructions.

As used herein, the term “protein” refers to a polymer of amino acids, peptide nucleic acids (PNAs) or mimetics, of no specific length and to all fragments, isoforms, variants, derivatives and modifications (glycosylation, phosphorylation, post-translational modifications, etc.) thereof.

As used herein, the terms “purified” and “to purify” and “purification” refers to the removal or reduction of at least one contaminant or non-desirous substance from a sample. For example, antibodies are purified by removal of contaminating non-immunoglobulin proteins. In another example, antibodies are purified by the removal of an immunoglobulin that does not specifically bind to the target molecule. The removal of non-immunoglobulin proteins and/or the removal of immunoglobulins that do not bind to the target molecule results in an increase in the percent of target-reactive immunoglobulins in the sample (e.g., “enrichment” of an antibody).

As used herein, the term “sample” is used in the broadest sense and can be obtained from any source in the body. A sample can encompass fluids, solids and tissues. In preferred embodiments, a sample can include one or more of the following fluids: serous fluid, urine, saliva, tears, blood, plasma, serum, aural fluid, nasal fluid, ear drainage, etc.

As used herein, the terms “specific binding” and “specifically binding” refer to the interaction between an antibody and an antigen.

As used herein the term “substrate” refers to any rigid or semi-rigid support to which molecules (e.g., nucleic acids, polypeptides, mimetics) may be bound. Examples of substrates include membranes, filters, chips, slides, wafers, fibers, magnetic, or nonmagnetic beads, gels, capillaries or other tubing, plates, polymers, and microparticles with a variety of surface forms including wells, trenches, pins, channels and pores.

A. Cerebrospinal Fluid

The present invention provides methods for detection and diagnosis of cerebrospinal fluid (CSF) leakage and associated conditions using antibodies that specifically bind to CSF-specific proteins. In particular, the present invention provides for monoclonal antibodies that specifically bind to CSF-specific proteins. Apparatus and methods for assaying and detecting CSF-specific proteins in a sample are also disclosed. Such apparatus can be used for inpatient (e.g., in surgery) and/or outpatient conditions (e.g., in clinic). In preferred embodiment, an easy-to-use apparatus can be operated by a physician or by any other individual. Prior experience is not necessary as the apparatus is simple to use and no special timing, dilutions or concentrations are required prior to using the apparatus herein, such that the same apparatus can be used to detect low and high concentrations of CSF-specific protein in a test sample.

CSF is produced by non-neural structures in the brain called “choroid plexus”. A choroid plexus is a collection of blood vessels with ion pumps that promote movement of water from the blood into the ventricles. These vessels have holes in their walls large enough to allow passage of water, ions (such as sodium and potassium), and small molecules (sugars and small proteins) through them while larger protein molecules (like albumin and immunoglobulin) and cells (red blood cells, white blood cells, platelets) cannot pass through the choroid plexus and remain in the blood. The CSF is thus normally a filtrate of the blood without cells or proteins.

Cerebrospinal fluid (CSF) plays a crucial role in the diagnosis and treatment of various disorders including, for example, infectious diseases, neoplastic processes, multiple sclerosis, demyelinating disorders, intracerebral hemorrhage, primary and metastatic malignancies, Alzheimer's disease, Creutzfeld-Jacob disease, global ischemia, psychiatric disorders, CSF otorrhea and rhinorrhea, as well as cortical and lacunar strokes. See Kleine, T. O., (2000) Fresenius J. Anal. Chem. 366(4): 382-6.

Diseases that break down the filtering mechanisms of the choroid plexus and/or the blood brain barrier (such as meningitis and some tumors) can allow blood cells to enter the spinal fluid which can be detected by a microscopic examination of a CSF sample, or by a spinal fluid tap assay (usually by lumbar puncture). On the other hand, detection of CSF leakage from the ventricles in the brain where it circulates is typically made using radiologic or laboratory methods. See Lund, V. J. et al., (2000) J. Laryngol. Otol. 114(12): 988-92 and Knight, J. A. (1997) Ann. Clin. Lab. Sci., 27(2): 93-104.

Examples of radiologic methods include CT and MRI scanning. CT and MRI scans can diagnose fractures and detect fluid levels within sinuses, thus suggesting cerebrospinal fluid leakage. Other radiologic techniques include contrast and radionuclide cisternography. Contrast cisternography can locate the site of leakage but may not detect intermittent leaks. Radionuclide cisternography is expensive, has a high false positive rate, and is not accurate in determining the location of leakage. Other methods include intrathecal fluorescein injection with visualization of the dye on nasal pads under a Wood's lamp. This is a sensitive method but also has a high false positive rate as any fluorescein which enters the circulation can cross the nasal mucosa. False positives can be reduced by comparison to pads placed in the oral cavity.

Laboratory methods for detection of CSF leakage include the halo sign test in which a nasal discharge is distinguished from cerebrospinal fluid by detection of a “halo sign” after blotting the sample on tissue paper, and measuring glucose, protein and/or chloride. The halo sign requires a large sample volume to produce and is not pathognomonic. Glucose and chloride are higher in cerebrospinal fluid than in nasal secretions while protein is lower. However, these levels can be altered by various factors, including abnormal serum concentrations and meningitis, and they have been shown to be nonspecific. Glucose oxidase test strips are positive in a majority of nasal and lacrimal secretions with cerebrospinal fluid range glucose levels present in approximately 25% of these samples. Glucose oxidase reagent strips should not be used to detect cerebrospinal fluid leakage despite their recommendation in various textbooks.

Cerebrospinal fluid has approximately one-tenth the amount of proteins as serum. Protein electrophoresis of cerebrospinal fluid shows a prominent albumin band. Albumin accounts for 55-70% of the total protein in cerebrospinal fluid. Other proteins that are also relatively small can cross the blood brain barrier and will also be detected on cerebrospinal fluid protein electrophoresis. These proteins include prealbumin, alpha-1 antitrypsin and transferrin. Prealbumin is also known as transthyretin since it binds thyroxine and vitamin A. Prealbumin is produced in both the liver and choroid plexus. It is present in serum only at low concentration and thus is not detected on serum protein electrophoresis. High molecular weight proteins such as haptoglobin, beta-lipoprotein, and complement do not normally cross the blood brain barrier and are usually not detected in cerebrospinal fluid by protein electrophoresis. Other proteins such as transferrin become modified in the CSF and can be distinguished in electrophoretic mobility and size from serum transferrin.

Transferrin is a 698 amino acid single chain glycoprotein with a molecular weight of approximately 80 kDa. The amino acid sequence of transferrin is listed in FIG. 10. Its N-terminal and C-terminal globular domains of transferrin each contain an iron-binding site which is involved in iron transport, particularly in serum transferrin. Furthermore, transferrin has two N-linked polysaccharide chains residing in the C-terminal domain at positions 413 and 611. These polysaccharide chains are branched with one or more terminal sialic-acid residues. The most common form of beta-1 transferrin, “tetrasialo-transferrin,” has four negatively charged sialic acid residues. Other forms of beta-1 transferrin include a mono-, di-, tri- and pent-sialo transferrin. The other transferrin isoform, beta-2 transferrin, is a desialylated form of the protein having no sialic acid residues. The protein exists in at least two isoforms, beta-1 and beta-2 transferrin, which can be distinguished by size and electrophoretic mobility depending on the number of sialic acid residues on the N-polysaccharide chains.

Unlike the other transferrin isoforms, beta-2 transferrin is normally present only in cerebrospinal fluid, aqueous and vitreous humors, and perilymph. See Kelly, R. H., et al. (2000) Clin. Chim. Acta. 288(1-2): 205-9. Beta-2 transferrin is generally not found in nasal fluids, aural fluids, saliva, tears or serum. Thus the detection of beta-2 transferrin in non-CSF, humor or perilymph fluid is an indication for CSF leakage.

In the central nervous system, neuraminidase causes desialation of part of the transferrin fraction. Loss of charged sialic acid groups changes the migration of this fraction on electrophoresis producing both beta-1 and beta-2 migrating bands. Additional isoforms of transferrin found in cerebrospinal fluid are formed by the action of neuraminidase on the beta-1 isoform. Due to a lower sialic acid content, these isoforms are less negatively charged and move slower towards an anode. These isoforms which migrate in the beta-2 region and are collectively known as “beta-2 transferrin.” On the other hand, isoforms that migrate in the beta-1 region are collectively known as “beta-1 transferrin.”

Serum samples from alcoholics and those with genetic transferrin variations may contain sialic acid depleted transferrin, giving rise to multiple transferrin bands on serum protein electrophoresis. This means that chronic alcoholics, people with lymphoma, and others with genetic transferring variations may have beta-2 transferrin present in the blood. Rare false positive cerebrospinal fluid leaks that may occur in these patients can be avoided by performing serum immunofixation electrophoresis after obtaining a positive result.

Until the present time, the only way to detect beta-2 transferrin in patients has been by immunofixation electrophoresis using transferrin antibodies that bind specifically to all transferrin isoforms. The beta-2 isoform is then resolved from the predominant serum isoform based upon its electrophoretic migration. Immunofixation electrophoresis to detect cerebrospinal fluid leakage was first reported in 1979 and its sensitivity and specificity is very high, being reported as 100%.

The immunofixation electrophoresis test using transferrin antibodies is performed in several laboratories across the United States. Samples are collected and sent to one of these laboratories for the test and 48 to 72 hours later the results are sent to the physician. The patient is hospitalized and monitored until the test results for cerebrospinal fluid leakage are determined. Thus, a patient who is suspected of having a cerebrospinal fluid leak must remain in the hospital until the diagnosis is confirmed or ruled out. sometimes resulting in unnecessary hospitalization.

The present invention relates to antibodies, methods and apparatus of detecting CSF-specific proteins. “CSF-specific proteins” refer to proteins that under normal healthy conditions are present in the CSF and possibly in the humor and/or perilymph, but which are not present significantly (or in similar amounts) in other bodily fluids such as blood, serum, tears, nasal discharge, ear drainage, saliva, urine, etc. Such proteins are referred to herein as CSF-specific proteins. Examples of CSF-specific proteins include beta-2 transferrin, CSF-tau, CSF-A42, and 14-3-3 proteins.

B. Antibodies

The present invention provides methods for producing antibodies and antibodies that specifically bind to CSF-specific proteins. Such antibodies are useful for detection of cerebrospinal fluid leakage and can be used for diagnosis of conditions associated with fluid leakage. Examples of antibodies include, but are not limited to, polyclonal antibodies, monoclonal antibodies, humanized antibodies, chimeric antibodies, single chain antibodies, Fab fragments, F(ab′)₂ fragments, fragments produced by FAb expression library, anti-idiotypic (anti-Id) antibodies or epitope-binding fragments of any of the above. Preferably, the antibodies are monoclonal antibodies.

Antibodies are prepared by first immunizing a suitable mammal with an antigen. Antigen can be obtained by any means known in the art. In one example, it is obtained by purification of a CSF-specific protein from cerebrospinal fluid, humor or perilymph. In another example, an antigen is obtained using recombinant protein expression that is post-translationaly modified by inserting an expression vector encoding a full-length CSF-specific protein or a fragment thereof into host cells. The cultivation of the cells in an appropriate medium can result in the production of full-length antigen or a partial fragment thereof, either as such or as a fusion protein in the cells or the culture supernatant. In one example, a human beta-2 transferrin antigen is obtained by removing all sialic acid residues of transferrin. Sialic acid residues can be removed using neuraminidase enzyme as further disclosed herein.

Once a CSF-specific protein (or fragment thereof) antigen is obtained, animals such as, for example, goats, rabbits, rats, mice, hamsters or humans may be immunized. Immunization is performed by administering to the animals the antigen, preferably along with an appropriate adjuvant such as complete Freund's adjuvant or a combination of aluminum hydroxide gel and pertussis vaccine. The antigen is administered by injection one to several times subcutaneously, intramuscularly, intravenously, into the foot pad or intraperitoneally. In general, immunization is performed one to ten times every one to four weeks from the initial immunization. Blood is collected at day 3-15 after each administration and the serum is examined for reactivity with the antigen by enzyme immunoassay (ELISA). The antibodies can then be isolated from the subject (e.g., from blood) and further purified using techniques, such as protein A chromatography, to obtain the IgG fraction.

At an appropriate time after immunization, such as one to 14 days after final immunization or when the antibody titers are at their highest, antibody-producing cells can be obtained from the subject and used for the preparation of monoclonal antibodies. Monoclonal antibodies are populations of antibodies that contain only one species of an antigen-binding site and are capable of immunoreacting with only one particular epitope. A monoclonal antibody composition, therefore, typically displays a single binding affinity for a particular polypeptide with which it immunoreacts.

The monoclonal antibodies of the present invention can be prepared by conventional methods known in the art, such as by fusing individual antibody producing cells (typically a splenocutes) from an immunized animal with cells derived from an immortal B lymphocyte tumor (typically a myeloma) to produce a hybridoma. See Köhler and Milstein et al. See Nature, 256:495-497(1975). The antibody producing cells are those obtained from a spleen, lymph node, bone marrow or tonsil of an animal. More preferably, antibody-producing cells are obtained from an animal's spleen.

The myeloma cells are those incapable of producing auto-antibody are derived from mammals, preferably from a mouse, rat, guinea pig, hamster, rabbit or a human, but more preferably from a mouse, rat or human. In a preferred embodiment, an established cell line from a mouse or a rat is used as a myeloma cell. Examples of myeloma cells include cell lines P3-X63Ag8-U1 (P3-U1) (Curr. Topics Microbiol. Immunol., 81, 1 (1978)) and P3-X63-Ag8 (X63) (Nature, 256:495 (1975)). In a preferred embodiment, SP2/0-AG14 myeloma cells are used for hybridoma construction (ref de St. Groth S F, Scheidegger D. J. Immunol. Methods 35: 1-21, 1980.). These cell lines can be subcultured in 8-azaguanine medium which is RPMI-1640 medium supplemented with glutamine (1.5 mM), 2 mercaptoethanol (5×10⁻⁵M), gentamicin (10 ug/ml) and fetal calf serum (FCS) (CSL, 10%), which is further supplemented with 8-azaguanine (15 ug/ml). Such cell lines should be subcultured in a normal medium 3-4 days before cell fusion to ensure a cell count of at least-2×10⁷ cells on the day of the cell fusion. Other techniques for producing hybridomas include the human B cell hybridoma technique described in Kozbor et al. (1983) Immunol. Today, 4:72; and the EBV-hybridoma technique and the trioma techniques.

After cloning the hybridoma, culture supernatants of the resulting hybridoma cells are screened to identify hybridomas that produce a monoclonal antibody which binds specifically to one or more of the CSF-specific proteins. In one embodiment, monoclonal antibodies can be screened to identify hybridomas that produce monoclonal antibody which binds specifically to beta-2 transferrin, an isoform of transferrin found only in CSF, humor and perilymph fluid, but which does not bind specifically to beta-1 transferrin isolated from other bodily fluids such as blood, urine, tears, mucus, lymph, puss or vitreous fluid.

Screening of hybridomas can be performed by culturing the hybridomas on a microtiter plate, for example, and then measuring its reactivity to human beta-2 transferrin used for the immunization. The screening assay can be performed by RIA or by enzyme immunoassay such as ELISA. In one embodiment a mouse or rat beta-2 transferrin monoclonal antibody producing hybridoma is selected according to the methods described in “Antibodies,” A Laboratory Manual, Cold Spring Harbor Laboratory, Chapter 14 (1988). An appropriate plate is coated with the antigen or a recombinant protein such as a fusion protein of the antigen. The plate is then reacted with a primary antibody, which may be the hybridoma culture supernatant or the purified antibody obtained from the hybridoma supernatant. The plate is then reacted with a secondary antibody, which may be an anti-species antibody, such as an anti-mouse or an anti-rat immunoglobulin antibody. The anti-species antibody binds specifically to the species from the first antibody. The secondary antibody is further labeled with, for example, biotin, an enzyme, a chemiluminescent substance or a radioactive compound. Subsequently a reaction is performed in accordance with the specific kind of label, whereby a hybridoma that is reactive specifically with the antigen (e.g., any CSF-specific protein) is selected as a hybridoma producing a mouse or rat monoclonal antibody. A cell exhibiting a stable high antibody titer is selected as a hybridoma cell line, which produces a monoclonal antibody. Such monoclonal antibodies can be used to detect and diagnose CSF leakage.

Similar selection process applies for selection of humanized antibodies, single chain antibodies or disulfide stabilized antibodies. Plates are coated with an antigen or a recombinant protein such as a fusion protein of the antigen. The plate is then reacted with a primary antibody. If the primary antibody is obtained from culture supernatant of transformants producing a humanized antibody, a single chain antibody, a disulfide stabilized antibody or an antibody purified therefrom, an antihuman immunoglobulin is used as a secondary antibody. The secondary antibody can be labeled with biotin, an enzyme, a chemiluminescence substance or a radioactive compound and a reaction is performed in accordance with the specific kind of the label for detection.

Any one of the hybridoma culture supernatant, the culture supernatant of the transformants producing a humanized antibody, single chain antibody or a disulfide stabilized antibody, or an antibody purified thereof can be mixed and reacted with labeled CSF-specific proteins, for example, with biotin, an enzyme, a chemiluminescent substance or a radioactive compound. Subsequently a reaction is performed according to the specific type of label so as to determine the activity and/or binding by the antibodies to the antigen.

Preparation of a monoclonal antibody from the hybridoma is performed by culturing the hybridoma in vitro or by placing the hybridoma in vivo in ascites of a mammal such as, for example, a mouse, rat, guinea pig, hamster, or rabbit. The monoclonal antibodies are then isolated from the culture supernatant or from the ascites of the mammal. When culturing the hybridoma in vitro, one may use any culturing methods and nutrient mediums known in the art or modifications thereof depending on the characteristics of cells and purpose of study. Hybridoma medium should be able to proliferate and maintain the hybridoma and to produce monoclonal antibody in culture supernatant.

Isolation and purification of the monoclonal antibody from the above-mentioned culture supernatant or ascites can be performed by applying saturated ammonium sulfate preciptiation, euglobulin precipitation, the caproic acid method, the caprylic acid method, ion exchange chromatography, or affinity column chromatography such as anti-immunoglobulin column chromatography and protein A column chromatography, and so forth.

In one embodiment of the present invention, 8-10 week-old mice or nude mice are treated, or more preferably administered intraperitoneally, with pristane (2,6,10,14-tetramethylpentadecane, 0.5 ml). The mice are then bred for approximately two weeks before they are administered intraperitoneally with the beta-2 transferrin monoclonal antibody-producing hybridoma cell lines disclosed herein. The cell lines are administered in an amount of approximately 2×10⁷ to 5×10⁶ cells per mouse. Ascites are then collected from the mouse and centrifuged at about 3,000 rpm, for 5 minutes to remove a solid portion. The precipitate is salted out and applied to a column for a caprylic acid precipitation, or a DEAE-Sepharose column, a protein A-column or a Cellulofine GSL2000 column (from Biochemical Industry) to collect IgG or IgM fractions. These fractions can be used as a purified monoclonal antibody. Subclass of the antibody can be determined using a monoclonal antibody typing kit. The mass of the protein can be calculated by a Lowry method or from the absorbance at 280 nm.

Isolated monoclonal antibodies which bind specifically to a CSF-specific protein, such as beta-2 transferrin, are useful in the diagnosis and prognosis of cerebrospinal fluid leakage and conditions associated therewith. For example, antibodies that bind specifically to beta-2 transferrin but which do not bind to beta-1 transferrin can be used to detect CSF when detection is made outside the CSF, humor or perilymph.

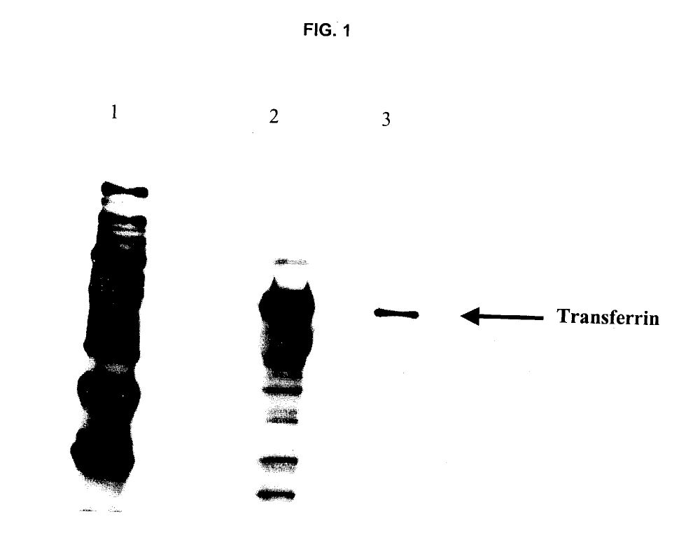

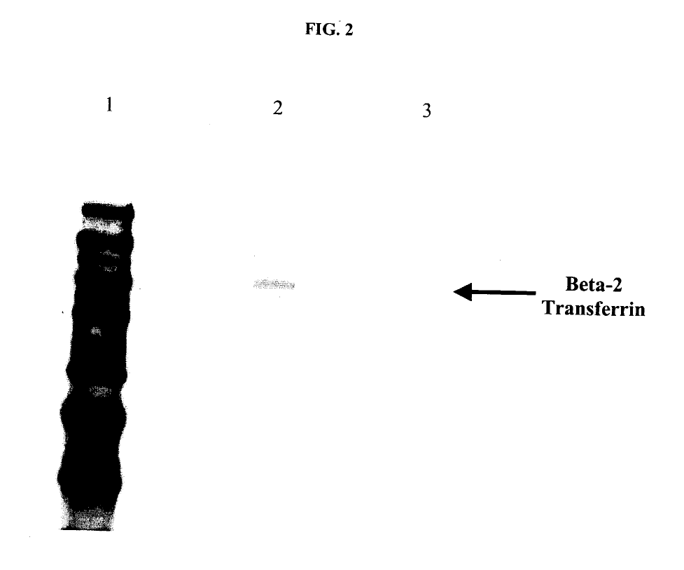

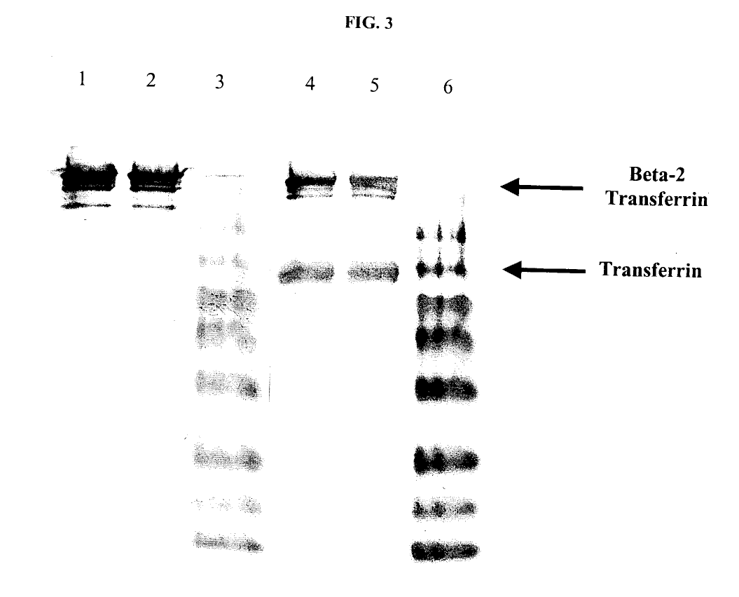

FIG. 1 illustrates binding of commercial transferrin antibody to cerebrospinal fluid (column 2) and to purified human transferrin (column 3). FIG. 2 illustrates binding of beta-2 transferrin antibody to cerebrospinal fluid (column 2) and purified human transferrin (column 3). Column 1 of both FIGS. 1 and 2 are molecular weight markers. As these figures illustrate, the commercial transferrin antibody binds to numerous proteins within the cerebrospinal fluid. See FIG. 1, column 2. On the contrary, beta-2 transferrin antibody generates only one band indicating that it binds specifically to only beta-2 transferrin. See FIG. 2, column 2. Furthermore, while the transferrin antibody binds to transferrin, the beta-2 transferrin antibody does not. See FIG. 2, column 3. This illustrates that beta-2 transferrin not only binds to beta-2 transferrin it also does not bind to transferrin. FIG. 3 illustrates an immunoassay between CSF and beta-2 transferrin in columns 1 and 2 and with transferrin in columns 4 and 5. Beta-2 transferrin antibodies of the present invention bind specifically to beta-2 transferrin in CSF, as is illustrated in columns 1 and 2 while commercially available transferrin antibodies bind to all transferrin isoforms within the CSF.

It is possible that when a non-human animal-derived monoclonal antibody is administered to a human, the antibody may be recognized as foreign matter such that an antibody against the non-human animal-derived monoclonal antibody is produced by the human body. This may result in side effects (J. Clin. Oncol., 2:881 (1984); Blood, 65: 1349 (1985); J. Natl. Cancer Inst., 80:932 (1988)) or in a reduced therapeutic effect (J. Immunol., 135:1530 (1985); Cancer Res., 46:6489 (1986)).

In order to avoid such problems, a non-human animal-derived monoclonal antibody may be converted to human chimeric antibodies or human CDR-grafted antibodies (reconstituted human antibodies) using, for example, gene recombinant technology. A human chimeric antibody is an antibody of which the variable region (“V region”) is derived from a non-human animal antibody and the constant region (“C region”) is derived from a human antibody. See Proc. Natl. Acad. Sci., 86:4220 (1984). When a human chimeric antibody is administered to a human, antibodies are hardly produced against the non-human animal derived monoclonal antibody and its half-life in blood is increased by a factor of six. See Proc. Natl. Acad. Sci., 86:4220 (1989). A human CDR-grafted antibody is a human antibody of which the CDR (complementarity determining region) is replaced with the CDR of a nonhuman animal-derived antibody. See Nature, 321:522 (1986). Furthermore, it has been reported with experiments on monkeys that a human CDR-grafted antibody has a lower immunogenicity, with the half-life in blood being increased by a factor of 4 to 5 compared with a mouse antibody. See J. Immunol., 147:1352 (1991). Thus a humanized beta-2 transferrin antibody is expected to reduce or completely eliminate the production of antibodies against itself as well as reduce or completely eliminate any side effects resulting from a therapeutic treatment.

Humanized antibodies can be produced by preparing a humanized antibody expression vector for expression in animal cells. A humanized antibody expression vector is a vector encoding CH and CL. “CH” refers to the constant regions of a human antibody heavy chain and CL refers to the constant region of a human antibody light chain. Any expression vector system that can express the constant domains in animal cells can be used, for example pAGE107 (Cytotechnology, 3:133 (1990); pAGE103 (J. Biochem., 101:1307 (1987); pHSG274 (Gene, 27:223 (1984); and pKCR (Proc. Natl. Acad. Sci., 78:1527 (1981). A promoter and enhancer can be used in an expression vector. Examples of promoters and enhancers include SV40 early promoter and enhancer (J. Biochem., 101:1307 (1987)); a Moloney mouse leukemia virus LTR promoter and enhancer (Biochem. Biophys. Res. Commun., 149, 960 (1987)); an immunoglobulin H chain promoter (Cell, 41:479 (1985)); and H chain enhancer (Cell, 33 717(1983)). The H and L chains may exist on the same expression vector or on separate vectors. In a preferred embodiment, both H and L chains exist on the same expression vector.

As a result of recent advances in protein and genetic engineering, smaller antibody molecules, such as single chain antibodies (Science, 242:423 (1988)) and disulfide stabilized antibodies (Molec. Immunology, 32:249 (1995)) may be prepared. Such antibodies usually have smaller molecular weights than monoclonal antibodies and humanized antibodies which allows them to enter into tissues more effectively.

The term “single chain antibody” generally refers to a polypeptide represented by formula VH-L-VL or VL-L-VH, wherein “VH” refers to a variable region in a heavy chain; “VL” refers to a variable region in a light chain and “L” refers to an appropriate peptide linker. Any single chain non-human beta-2 transferrin antibody or single chain human CDR-grafted antibody can be constructed by inserting the cDNAs encoding VH and VL of the non-human animal antibody or human CDR-grafted antibody into host cells. A host cell for use in expressing a single chain antibody can be selected from among E. coli, yeast, and animal cells. The single chain antibody can be secreted out of the cell and transported into the periplasm region or retained within the cell by inserting a cDNA encoding an appropriate signal peptide into the expression vector.

The term “disulfide stabilized antibody” refers to an antibody prepared by binding through a disulfide bond two polypeptides in which each one of the amino acid residues in VH and VL is replaced with cysteine residues. The amino acid residues to be replaced with cysteine residues can be selected on the basis of a presumed steric structure of an antibody in accordance with the method described by Reiter et al. Protein Engineering, 7:697 (1994). Eiryhrt non-human or human CDR-grafted antibodies can be used as VH and VL in the disulfide stabilized antibodies herein. Overall, the single chain and disulfide-stabilized antibodies can be used to increase therapeutic efficacy. See Cancer Research, 55:318 (1995).

Any of the antibodies herein can be used as a detection antibody by labeling the antibody with a reporter or detectable reagent. A “reporter” or “detectable reagent” refer to any substance which can be attached to specific binding agents, such as antibodies or antigens, which is capable of producing a signal that is detectable by visual or instrumental means. Various suitable labels for use in the present invention can include chromogens, catalysts, fluorescent compounds (such as, for example, fluorescein, phycobiliprotein, rhodamine), chemiluminescent compounds, radioactive elements, colloidal metallic (such as gold), non-metallic (such as selenium) and dye particles, enzymes, enzyme substrates, and organic polymer latex particles, liposomes or other vesicles containing such signal producing substances, and the like. Examples of enzymes that can be used as labels include phosphatases and peroxidases, such as alkaline phosphatase and horseradish peroxidase which are used in conjunction with enzyme substrates, such as nitro blue tetrazolium, 3,5′,5,5′-tetranitrobenzidine, 4-methoxy-1-naphthol, 4-chloro-1-naphthol, 5-bromo-4-chloro-3-indolyl phosphate, chemiluminescent enzyme substrates such as the dioxetanes.

Preferably a CSF-specific antibody is conjugated to colloidal gold or other detectable reagent for visualization and/or quantification. In alternative embodiments, such antibody may be conjugated to an enzyme moiety that typically yields a colored or fluorescent reaction product following the addition of a suitable substrate. Commonly used conjugated enzymes include horseradish peroxidase, urease, alkaline phosphatase, glucoamylase and beta-galactosidase.

The linking of labels, e.g., labeling of peptides and proteins, is well known to those of ordinary skill in the art. For example, monoclonal antibodies produced by a hybridoma can be labeled by metabolic incorporation of radioisotope-containing amino acids provided as a component in the culture medium. See, for example, Galfre et al., (1981) Meth. Enzymol., 73: 3-46. The techniques of protein conjugation or coupling through activated functional groups are particularly applicable. See, Avrameas et al., (1978) Scand. J. Immunol., 8(7): 7-23. Rodwell et al. (1984) Biotech., 3: 889-894 and U.S. Pat. No. 4,493,795.

C. Apparatus and Methods of Detecting Beta-2 Transferrin

The present invention provides apparatus and methods of detecting CSF leakage using one or more antibodies that specifically binds to at least one CSF-specific protein. Such antibodies may be used alone or in combination with other antibodies (e.g., species specific antibodies) in an immunoassay to detect the presence or absence of a CSF-specific protein. An “immunoassay” refers to any method of detection or assay that uses at least one specific antibody for the detection or quantification of an antigen. Examples of immunoassays include but are not limited to, Western blots, ELISAs, radio-immunoassays, and immunofluorescence assays. Furthermore, many different ELISA formats are known to those in the art, any of which will find use in the present invention. It is not intended that the present invention be limited to these assays.

In addition, other antigen-antibody reactions may also be used. Such reactions include, for example, “flocculation” (i.e., a colloidal suspension produced upon the formation of antigen-antibody complexes), “agglutination” (i.e., clumping of cells or other substances upon exposure to antibody), “particle agglutination” (i.e., clumping of particles coated with antigen in the presence of antibody or the clumping of particles coated with antibody in the presence of antigen), “complement fixation” (i.e., the use of complement in an antibody-antigen reaction method), and other methods commonly used in serology, immunology, immunocytochemistry, immunohistochemistry, and related fields.

Immunoassays may be exemplified by a labeling agent includes enzymes, fluorescent materials, chemiluminescent materials, biotin, avidin or radioisotopes, etc., more specifically, enzymes such as peroxidase (for example, horseradish peroxidase), alkaline phosphatase, beta.-D-galactosidase, glucose oxidase, glucose-6-phosphate dehydrogenase, alcohol dehydrogenase, malate dehydrogenase, penicillinase, catalase, apo-glucose oxidase, urease, luciferase or acetylcholinesterase; fluorescent materials such as fluorescein isothiocyanate, phycobiliprotein, chelating compounds of rare-earth metals, dansyl chloride or tetramethylrhodamine isothiocyanate; radioisotopes such as ³H, ¹⁴C, ¹²⁵I or ¹³¹I; biotin; avidin; or chemiluminescent materials. Radioisotopes and fluorescent materials, even when used alone, give a detectable signal. On the other hand, enzymes, chemiluminescent materials, biotin, and avidin give no detectable signals, when used alone. In these cases, one or more substances are needed with the substances in order to give a detectable signal. For example, when the substance is an enzyme, at least a substrate for the enzyme is necessary to give a detectable signal. Various types of substrates are selectable depending on the methods for measuring the enzyme activity (colorimetry, immunofluorescence method, bioluminescence method or chemiluminescence method, etc.). For example, hydrogen peroxide is used as a substrate for peroxidase. When biotin is selected, avidin or enzyme-conjugated avidin is used for the reaction with biotin generally but not always. According to needs, various coloring agents are further used for the reaction depending on the type of the substrate.

In some embodiments, a Western blot may be used to detect beta-2 transferrin in a sample. The term “Western blot,” “Western immunoblot” or “Western” refer to the immunological analysis of protein(s), polypeptides or peptides that have been immobilized onto a membrane support. The proteins are first resolved by polyacrylamide gel electrophoresis (i.e., SDS-PAGE) to separate the proteins, followed by transfer of the protein from the gel to a solid support, such as nitrocellulose or a nylon membrane. The immobilized proteins are then exposed to an antibody having reactivity towards an antigen of interest. Examples of antibodies having reactivity towards beta-2 transferrin include any of the antibodies herein (e.g., polyclonal antibodies, monoclonal antibodies, single chain antibodies, humanized antibodies, disulfide stabilized antibodies, etc.). In a preferred embodiment, monoclonal antibodies disclosed herein are utilized. The binding of the antibody (i.e., the primary antibody) is detected by use of a secondary antibody which specifically binds the primary antibody, or preferably to the antigen (beta-2 transferrin) and primary antibody complex. The secondary antibody is typically conjugated to an enzyme which permits visualization of the antigen-antibody complex by the production of a colored reaction product or catalyzes a luminescent enzymatic reaction (e.g., the ECL reagent, Amersham).

In another embodiment, the antibodies herein are detected using ELISA methods. As used herein, the term “ELISA” refers to enzyme-linked immunosorbent assay (or EIA). Numerous ELISA methods and applications are known in the art, and are described in many references. See, e.g., Crowther, “Enzyme-Linked Immunosorbent Assay (ELISA),” in Molecular Biomethods Handbook, Rapley et al. (eds.), pp. 595-617, Humana Press, Inc., Totowa, N.J. (1998); Harlow and Lane (eds.), Antibodies: A Laboratory Manual, Cold Spring Harbor Laboratory Press (1988); Ausubel et al. (eds.), Current Protocols in Molecular Biology, Ch. 11, John Wiley & Sons, Inc., New York (1994). In addition, there are numerous commercially available ELISA test systems.

One of the ELISA methods used in the present invention is a “direct ELISA,” where an antigen (e.g., beta A-MDA peptide) in a sample is detected. In one embodiment of the direct ELISA, a sample-containing antigen is exposed to a solid (i.e., stationary or immobilized) support (e.g., a microtiter plate well). The antigen within the sample becomes immobilized to the stationary phase, and is detected directly using an enzyme-conjugated antibody specific for the antigen.

In an alternative embodiment, an antibody specific for an antigen is detected in a sample. In this embodiment, a sample containing an antibody (e.g., a beta-2 transferrin antibody) is immobilized to a solid support (e.g., a microtiter plate well). The antigen-specific antibody is subsequently detected using purified antigen and an enzyme-conjugated antibody specific for the antigen.

In an alternative embodiment, an “indirect ELISA” is used. In one embodiment, an antigen (or antibody) is immobilized to a solid support (e.g., a microtiter plate well) as in the direct ELISA, but is detected indirectly by first adding an antigen-specific antibody (or antigen), then followed by the addition of a detection antibody specific for the antibody that specifically binds the antigen, also known as “species-specific” antibodies (e.g., a goat anti-rabbit antibody), which are available from various manufacturers known to those in the art (e.g., Santa Cruz Biotechnology; Zymed; and Pharmingen/Transduction Laboratories).

In other embodiments, a “sandwich ELISA” is used, where the antigen is immobilized on a solid support (e.g., a microtiter plate) via an antibody (i.e., a capture antibody) that is immobilized on the solid support and is able to bind the antigen of interest. Following the affixing of a suitable capture antibody to the immobilized phase, a sample is then added to the microtiter plate well, followed by washing. If the antigen of interest is present in the sample, it is bound to the capture antibody present on the support. In some embodiments, a sandwich ELISA is a “direct sandwich” ELISA, where the captured antigen is detected directly by using an enzyme-conjugated antibody directed against the antigen. Alternatively, in other embodiments, a sandwich ELISA is an “indirect sandwich” ELISA, where the captured antigen is detected indirectly by using an antibody directed against the antigen, which is then detected by another enzyme-conjugated antibody which binds the antigen-specific antibody, thus forming an antibody-antigen-antibody-antibody complex. Suitable reporter reagents are then added to detect the third antibody. Alternatively, in some embodiments, any number of additional antibodies are added as necessary, in order to detect the antigen-antibody complex. In some preferred embodiments, these additional antibodies are labeled or tagged, so as to permit their visualization and/or quantification.

In preferred embodiment, an immunoassay is accomplished using an apparatus that is capable of producing rapid results, such as a strip test. The apparatus of the present invention can have multiple shapes and forms. The apparatus are preferably bound with antibodies that specifically bind to one or more CSF-specific proteins, such as beta-2 transferrin. In a preferred embodiment, an apparatus comprises of one or more monoclonal antibodies that specifically bind a CSF-specific protein wherein each monoclonal antibody binds to a different epitope. Thus, the apparatus and methods herein can eliminate the need of the immunofixation electrophoresis tests, laboratory tests and radiologic tests.

In preferred embodiments, the apparatus and methods are easily adaptable for rapid, convenient use such that they can be operated by a physician or by any other individual without prior experience. Furthermore, the apparatus preferably requires no special timing, dilutions or concentrations prior to use. For example, the same apparatus can detect low and high concentrations of a CSF-specific protein in the test sample. The simplicity of use and quick results make the apparatus herein appropriate for use in surgery or in outpatient treatment at home by a patient or at any other setting. Furthermore, the methods and apparatus may be designed to give a simple yes/no determination of the presence of a CSF-specific protein in a test sample. In some preferred embodiment, a yes/no determination can be visualized by a color change or other physical change. In additional preferred embodiment, a determination can be obtained in less than 20 minutes, more preferably in less than 10 minutes, more preferably in less than 5 minutes, and more preferably in less than 1 minute.

The apparatus herein may be configured in any manner suitable for providing a test area. In one embodiment, the apparatus herein comprises of a lateral flow strip test, also known as immunochromatographic assay, or simply a strip test. Strip tests are preferably used in applications for home testing and for rapid point of care testing. Strip test technology offers a range of benefits including being user-friendly, relatively inexpensive and providing quick results. A lateral flow test strip is composed of two main regions: a first antibody region (also referred to as conjugate release pad) and a test region (also referred to as a second antibody region).

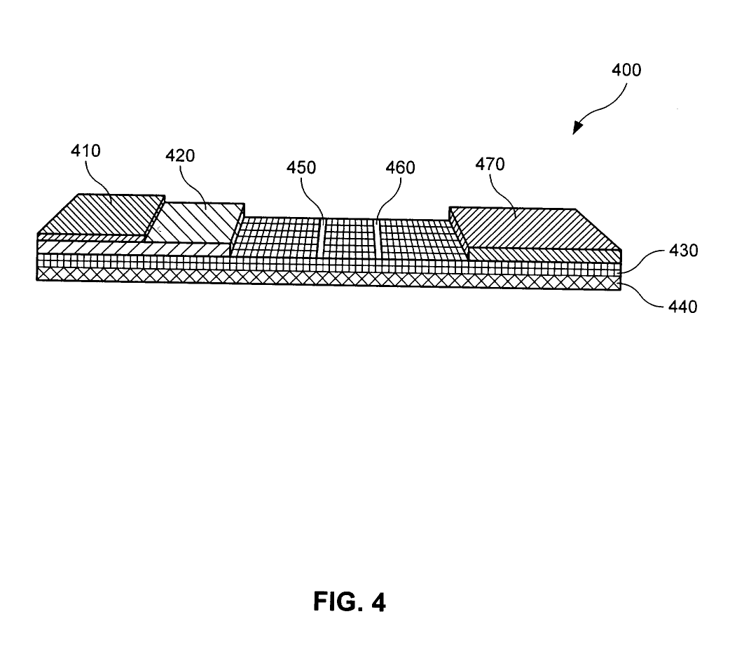

FIG. 4 illustrates another embodiment of a strip test. The strip test in FIG. 4 comprises of an application pad 410 in its proximal end, a conjugate release pad 420 contiguous to the application pad 410, a lateral flow membrane (also referred to as a predator membrane) 430 contiguous to the conjugate release pad and a backing 440 contiguous to the lateral flow membrane 430. The lateral flow membrane 430 further comprises one or more proximal testing regions 450 and optionally one or more distal control regions 460. The strip test may also comprise an absorbent pad 470, which is contiguous with the lateral flow membrane.

An application pad 410 can comprise of any material that allows for a flow-through of proteins and/or other molecules to be tested while filtering out any large particulate matter in a sample. An application pad 410 also functions to hold the sample so that it can slowly wick through into the conjugate release pad without overloading the test strip. In a preferred embodiment, an application pad 410 is composed of Hemasep™ V Medium. Hemasep™ V Medium is a modified polyester medium that can separate whole blood from plasma by chromatography. As test samples often have at least a trace amount of blood, a Hemasep™ V Medium is preferable. In another preferred embodiment, the application pad is composed of LoProsorb™ Medium.

Once a test sample (or portion thereof) has migrated through an application pad, it arrived at a conjugate release pad 420, situated between the application pad 410 and a lateral flow membrane 430. A conjugate release pad contains a detector antibody conjugated to a detectable reagent. A detector antibody can be any antibody that specifically binds to the antigen or CSF-specific protein, more preferably such antibody is a monoclonal antibody. In one embodiment, a detector antibody is an antibody that specifically binds to beta-2 transferrin, or more preferably a monoclonal antibody that specifically binds to beta-2 transferrin. A detector antibody is preferably conjugated to a detectable reagent that can be visualized with the naked eye and quantified. In a preferred embodiment a detector antibody is conjugated to colloidal gold. In some embodiments, a conjugate release pad is composed of an Accuwik™ Membrane. If a sample loaded onto the strip test contains an antigen (e.g., beta-2 transferrin), a colloidal gold conjugated detector antibody will bind to it and allow for its detection.

From a conjugate release pad, a sample (or portion thereof) migrates to a lateral flow membrane 430. The sample enters the membrane and moves towards the distal end (adsorbent pad) of the strip test via capillary action. A lateral flow membrane can comprise of any substance that allows for the flow-through of molecules especially proteins and antibodies. In a preferred embodiment, the lateral flow membrane is a nitrocellulose membrane.

Approximately half way between the proximal and distal ends of the lateral flow membrane are one or more testing regions 450. The testing regions comprise of immobilized capture antibodies or secondary antibodies. The capture antibodies can specifically bind the antigen (beta-2 transferrin) or the antigen-detector antibody complex (beta-2 transferrin-detector antibody). By binding to the antigen or antigen-detector antibody complex, the capture antibody triggers a change in appearance in testing region 450 which can be visualized and preferably quantified, for example, by a change in pattern or color. In a preferred embodiment, the capture antibody binds specifically to the antigen at a different epitope than the detector antibody. For example, a capture antibody can be an antibody that can specifically bind to beta-2 transferrin at a different epitope than a detector antibody. The detector antibody and the capture antibody can be any of the antibodies disclosed herein, preferably monoclonal antibodies. The presence of a pattern or color at the testing region 450 is an indication of the presence of the CSF-specific protein in the test sample—the absence of such a pattern is an indication of a lack of such protein.

In general, the amount of polypeptide or antibody immobilized on the membrane (e.g., in the test regions or control regions) is selected to generate a visually discernible pattern when the biological sample contains a level of antibodies that would be sufficient to generate a positive signal in an ELISA. Preferably, the amount of polypeptide immobilized on the testing region ranges from about 25 ng to about 1 ug, and more preferably from about 50 ng to about 500 ng.

The apparatus herein can further optionally include one or more control regions 460. A control region 460 can comprise of any substance that can serve to confirm the proper operation of the apparatus (e.g., by indicating the completion of the assay in the testing region). In preferred embodiments, the control region comprises of immobilized anti-species antibodies that will specifically bind to the detector antibody regardless of whether an antigen is present. Control regions 460 can also be used to provide a qualitative indication of the relative concentration of a CSF-specific protein in a sample tested, for example, by using a detectable reporters and a calibration curve to calibrate the amount of such protein.

The absorbent pad 470 is located at the distal end of the strip test. The absorbent pad 470 serves as a reservoir to hold the sample after it has wicked across the lateral flow membrane for a short period of time (roughly 15-20 minutes) before the sample begins to flow back across the membrane towards the proximal end. Finally, the strip test backing 440 serves to hold the above components in place.

In a variation of the apparatus herein, the lateral flow membrane further comprises a filtering region situated between the conjugate release pad and the testing region. The filtering region serves to slow the migration of CSF-specific proteins (antigens) from the sample that did not bind the labeled detector antibody and limit their ability to reach the testing region before the bound antigen. This reduces the possibility that non-bound antigen will saturate the binding sites in the test region.

In a variation of the test strip above, the control region 460 is composed of immobilized CSF-specific antigen (e.g., beta-2 transferrin). The introduction of a sample to the strip test results in the migration of at least some unbound detector antibody from the conjugate release pad 420 to the control region 460 where the conjugated detector antibody binds the immobilized antigen and creates a visual appearance (e.g., a color change).

In another variation of the above embodiment, the apparatus includes a first control region 462 and second control region 462, distal to the first control region 460. The first control region contains a detector antibody. This detector antibody does not necessarily have to bind CSF-specific proteins but must be conjugated to a detectable reagent. The second control region 462 contains an immobilized capture antibody that specifically binds the detector antibody of the first control region. The introduction of a fluid sample to the test strip results in the migration of detector antibody from the first control region towards the absorbent pad resulting in a formation of a first control detector antibody-second control capture antibody complex, which can be visually detected.

In another variation of the strip test apparatus herein, the conjugate release pad comprises of more than one detector antibody conjugated to a detectable reagent. Preferably, each detector antibody binds to a different epitope of a CSF-specific antigen. More preferably, the antibodies are monoclonal antibodies. A strip test having more than one detector antibody can optionally have more than one testing region. Preferably, multiple testing regions are adjacent to one another either laterally or transversely in the lateral flow membrane.

The apparatus and methods herein are preferably highly sensitive to CSF-specific proteins and produce results with high degree of accuracy. For example, the apparatus and methods herein can detect a concentration of beta-2 transferrin that is between 5 ng/mL and 250 μg/mL in a test sample, or more preferably at least about 5 ng/mL. Furthermore, the apparatus and methods herein are designed to avoid the production of false positives through the use of antibodies that are highly specific for human beta-2 transferrin and that do not bind beta-i transferrin. FIG. 3 illustrates the specificity of the antibodies disclosed herein to human beta-2 transferrin. Column 1 illustrates molecular weight markers for commercial transferrin antibodies and beta-2 transferrin antibodies as disclosed herein. Column 2 illustrates binding of commercial transferrin antibodies to cerebrospinal fluid and beta-2 transferrin antibodies to cerebrospinal fluid. Beta-2 transferrin antibody specifically binds to beta-2 transferrin and does not bind to beta-1 transferrin or any other polypeptides in the cerebrospinal fluid. On the other hand, commercial transferrin antibodies bind to a plethora of proteins such that it is difficult to detect the presence or absence of beta-2 transferrin in a sample. Finally, column 3 illustrates specific binding of transferrin antibody to transferrin polypeptide and no reaction between beta-2 transferrin antibody and transferrin.

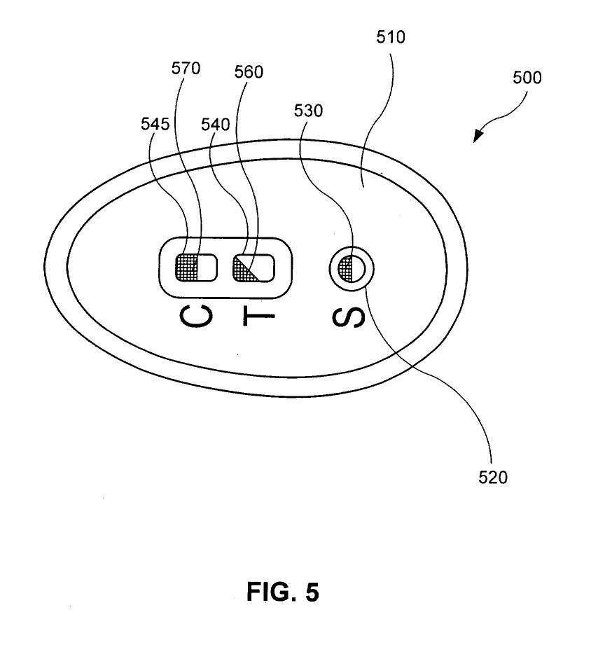

In another embodiment, a strip test apparatus is located inside a housing or other solid support (such as, for example, a plastic housing). This embodiment is especially useful for samples that are hard to obtain and FIG. 5 illustrates one example of such an apparatus 500 wherein the housing 510 having a hole 520 located over the application pad 530. The housing can also have windows 540 and 545 covering the testing region(s) 560 and control region(s) 570, respectively. In some embodiments, windows 540 and 545 are one and the same window. A section of the application pad 530 can be seen through the hole 520 and a section of the testing region(s) 560 and control region(s) 570 can be seen through their respective windows, 540 and 545, or one window.

A sample obtained from a patient can be contacted with the application pad 530 using a micropipette or any other mechanism. The sample then migrates to a conjugate release pad having a first antibody (detector antibody) that can specifically bind to a CSF-specific protein (a CSF-specific antibody). The first antibody is conjugated to a detectable reagent, preferably colloidal gold. As the sample migrates through the conjugate release pad, CSF-specific antibodies in the pad specifically bind CSF-specific proteins in the sample. The sample then migrates to the lateral flow membrane having testing region(s) 560 and control region(s) 570, distal to the testing region(s). The testing region(s) 560 in the lateral flow membrane have immobilized to them antibodies that can specifically bind to CSF-specific proteins or CSF-specific antibodies bound to CSF-specific proteins. As the antibodies in the test region are immobilized, labeled detector antibodies bound to CSF-specific proteins will also become immobilized resulting in a visible change within the window 540 due to the aggregation of detectable reagents. Sample that is not immobilized in the testing region 560 will continue to migrate to the control region 570. A control region can comprise of an immobilized anti-specie antibody that can specifically bind to detector antibody bound to a CSF-specific protein or not. The immobilization of detector antibody in the control region will result in a color change within window 545. Preferably, an absorbent pad is located distal to the control region. An absorbent pad inside housing 510 may be situated distal to the control region(s) 660 to reduce the possibility of backwards flow of sample through the lateral flow membrane.

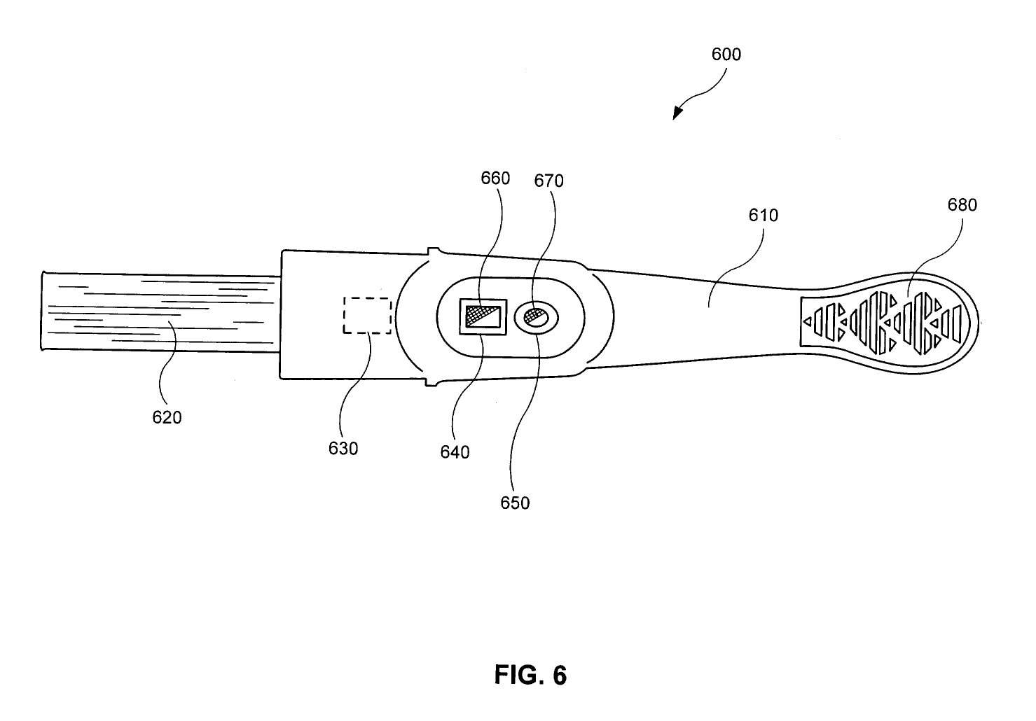

In yet another embodiment illustrated by FIG. 6, the detection apparatus 600 is modified for direct contact with a sample. In this embodiment, an application pad 620 is partially external in its proximal end to a housing 610 such that the proximal end can be used to directly contact a tissue sample. This embodiment is especially useful in surgery or when a sample is easily accessible (e.g., nose discharge). After the apparatus 600 is contacted with the sample, the sample migrates from the proximal end of the application pad 620 into the housing 610 where it reaches a conjugate release pad at location 630 within the housing 610.

The conjugate release pad comprises a first antibody (detector antibody) that can specifically bind to a CSF-specific protein (a CSF-specific antibody). The first antibody is conjugated to a detectable reagent, preferably colloidal gold. As the sample migrates through the conjugate release pad, CSF-specific antibodies in the pad specifically bind CSF-specific proteins in the sample. From the conjugate release pad, the sample migrates to a lateral flow membrane having testing region(s) 660 and control region(s) 670 distal to the testing region(s), and visible through respective windows 640 and 650.

The testing region(s) 660 in the lateral flow membrane have immobilized to them antibodies that can specifically bind to CSF-specific proteins or CSF-specific antibodies bound to CSF-specific proteins. As the antibodies in the test region are immobilized, labeled detector antibodies bound to CSF-specific proteins will also become immobilized resulting in a visible change within the window 640 due to the aggregation of detectable reagents. Sample that is not immobilized in the testing region(s) 660 will continue to migrate to the control region(s) 670. A control region can comprise of an immobilized anti-specie antibody that can specifically bind to detector antibody bound to a CSF-specific protein or not. The immobilization of detector antibody in the control region will result in a color change within window 650. Preferably, an absorbent pad is located distal to the control region within housing 610 to prevent the backflow of fluid sample. The solid support can optionally have a holding area 680 for an easier grip.

The apparatus and methods herein can be utilized to diagnose cerebrospinal fluid leakage by detecting the presence of one or more CSF-specific proteins in a sample of blood, serum, tears, saliva, nasal discharge, ear discharge, or any other tissue or bodily fluid aside from cerebrospinal fluid, humor and perilymph. Furthermore, as cerebrospinal fluid leakage is associated with various conditions including, but not limited to, rhinorrhea, otorrhea, recurrent meningitis, celebrities, chronic headaches, neck aches, loss of hearing, etc. (see Patel, R. B. et al. (2000) Ear Nose throat J 79(5):372-2, 376-8), the present invention also provides for a diagnosis of conditions associated with cerebrospinal fluid leakage.

In preferred embodiments, detection of CSF-specific proteins is made by contacting a test sample with an application pad (e.g. 410, 510, 610). A test sample can comprise of any tissue, solid, or fluid, including but not limited to blood, plasma, culture supernatant or centrifugation supernatant, urine, saliva, each discharge, tears, nasal fluid, aural fluid and excluding CSF, humor and perilymph fluid. The sample does not need to be diluted or concentrated before applying it to the pad. In some embodiment, a tissue sample or fluid can be directly contacted with application pad 610. This is especially useful in surgery or when there is nose or ear discharge. In other embodiment, a test sample is obtained from a patient and is applied to an application pad 510 inside a window 520 using a pipette.

As the methods and apparatus herein are easy to use, any person including a patient can perform the immunoassays. Furthermore, the ease of use allows these assays to be preformed at any location including at home, in clinic or in operation. For example, the immunoassays herein can be performed immediately after a trauma or a head injury. The can also be preformed during-surgery or post-surgery, especially in head and brain surgery. Furthermore, the present invention contemplates the repetitive use of the methods and immunoassays herein to detect its onset or recurrence, especially in testing individuals previously diagnosed with CSF leakage, those who are suspected of having CSF leakage, and those who are at risk of developing CSF leakage. Individuals suspected of having cerebrospinal fluid leakage include those having recently experienced trauma or showing symptoms such as headaches, nose aches, loss of hearing etc. Such individuals can be tested (or perform self-evaluation) using the apparatus and methods herein daily, weekly, monthly, quarterly, or bi-annually. A self-evaluation can be as simple as placing an application pad 610 inside a nostril and observing a color change (or lack thereof) in the test region 660. It is further contemplated that the detection methods and apparatus herein can be used to monitor CSF leakage to make determination regarding treatment of a patient. Treatments for CSF leakage include, for example, CSF diversion through lumbar drain and primary surgical repair.

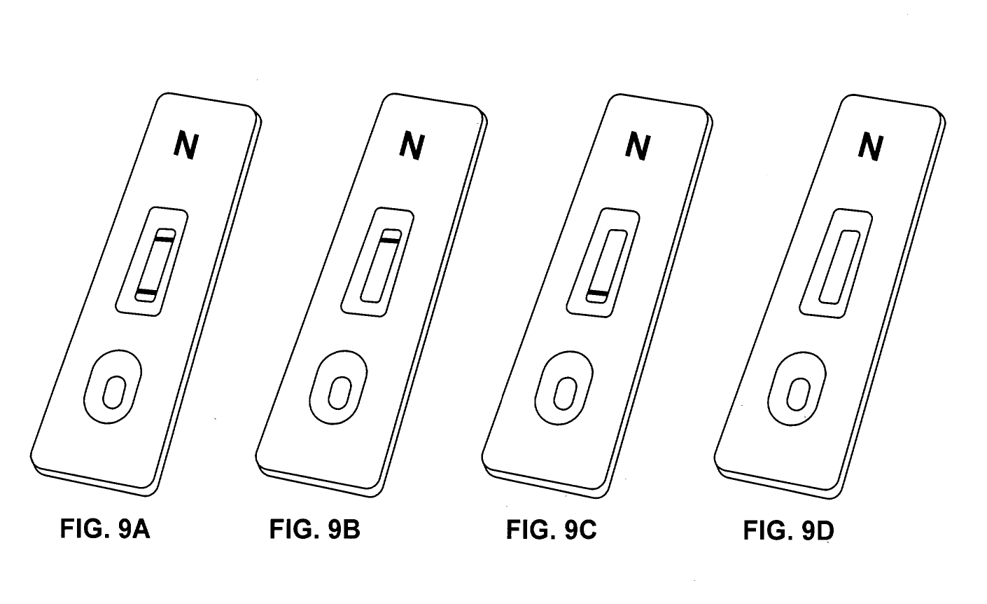

When reading a test assay using the apparatus herein, even the faintest visible change should be considered a result. There are four possible outcomes that can be observed using the apparatus herein. FIG. 9 illustrates the four possible outcomes. A first possible outcome, illustrated in FIG. 9A, is that two lines appear (one in the testing region and one in the control region). This indicates a positive assay and may be a diagnosis for CSF leakage and conditions associated therewith. The second possible outcome is illustrated in FIG. 9B, is a single line in the control region. This may be a valid negative result. A third possible outcome, illustrated in FIG. 9C, is a positive test line but no control line. This indicates a faulty assay and requires rerunning the assay on a new strip test. A fourth possible outcome, illustrated in FIG. 9D, is that no lines appear. This may also be the result of a faulty assay and a new assay should be conducted.

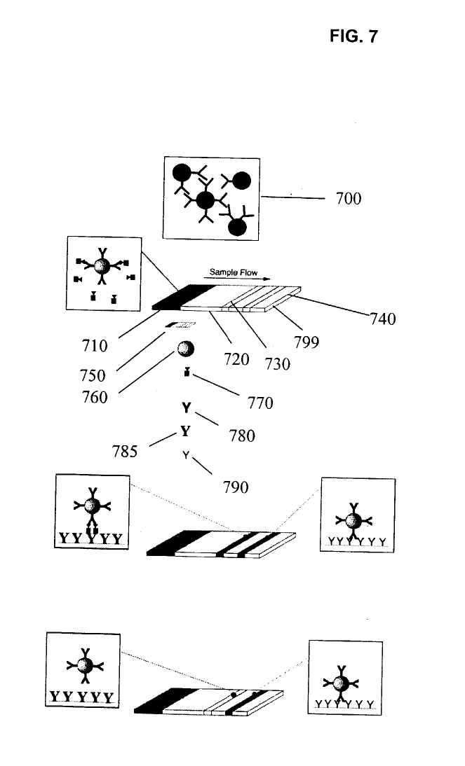

FIG. 7 illustrates an embodiment of the methods herein. Sample 700 is obtained from an individual to be tested for CSF leakage. A sample can comprise of tissue and/or bodily fluids including but not limited to blood, plasma, nasal secretions, sinus fluid, aural fluids, serum, tears, saliva, ear drainage, etc. In many cases a sample will be a mixture of bodily fluids and tissue, often including blood. The sample 700 is contacted with a proximal end 710 of a rapid test apparatus 750 wherein the proximal end contains a first antibody 770 that can specifically bind to the antigen 770, a CSF-specific protein, at an epitope 780. The CSF-specific protein 760 having antigen 770 and other unbound proteins than migrate along the lateral flow membrane or solid support 720 via capillary action to one or more test regions 730. A test region 730 comprises of a second antibody 785 that is immobilized to the lateral flow membrane or solid support 720. The second antibody 785 can specifically bind either the antigen (CSF-specific protein) 760 or the antigen-first antibody complex 760-770. Preferably, the second antibody 785 can bind antigen 760 at a different epitope 780 than the first antibody 770. Either the first antibody 770 or second antibody 785 is preferably labeled with a detectable reagent, such that binding can be detected visually. For example, if the first antibody 770 is conjugated with colloidal gold, upon binding of the antigen-first antibody complex 760-770 to the test region 730 a color change can be visualized.

Furthermore, a detection device 750 may have one or more control regions 740. A control region 740 is located distal to the test regions(s) 740 on the lateral flow membrane or solid support 720. The control region can comprise of any substance that will allow detection of a completed assay (migration over the test region). In one embodiment, a control region 740 comprises a third antibody 790 that can detect CSF-specific proteins 750 that are not bound to a first antibody 770 and are not immobilized to a test region 775. Such antibodies are preferably anti-species antibodies. In a preferred embodiment, the primary antibodies 770 are monoclonal antibodies.

Reagents and/or the apparatus herein may be combined into kits. The present invention contemplates the use of kits for the diagnosis of cerebrospinal fluid leakage and conditions associated therewith. In one embodiment, a kit comprises a first antibody that binds specifically to CSF-specific proteins (e.g., beta-2 transferrin). Preferably, such first antibody is a monoclonal antibody. A kit may further comprise a second antibody that binds specifically to the same CSF-specific proteins or to a complex of the first antibody CSF-specific protein complex. In some embodiments, the second antibody may be a species-specific antibody, which binds specifically to the first antibody of the kit. Furthermore, a third antibody may be provided within the kit. In some embodiments, such third antibody may be a species-specific antibody that can bind specifically to the first antibody or the second antibody, or both. In other embodiments, the third antibody can bind specifically to the CSF-specific protein.

In any of the embodiments herein, the first antibody, second antibody, third antibody, a combination of the above antibodies or all of the above antibodies are immobilized on a substrate.

In any of the embodiments herein, the first antibody, second antibody, third antibody, a combination of all of the above antibodies or all of the antibodies are labeled. A label can be in a separate container within the kit or can be conjugated to the first and/or second antibodies. In a preferred embodiment, a detectable reagent comprises of colloidal gold and is conjugated to the first antibody.

Kit reagents can optionally include buffers, such as dilution buffers and wash buffers. In one embodiment a buffer solution contains PBS, Triton X-100 and sodium azide. The PBS serves to adjust the sample to a neutral pH of 7 such that the antibodies will be able to function properly. The Triton X-100 is a surfactant that helps prevent aggregates from forming and blocking the flow across the lateral flow membrane. Sodium azide is a general preservative and disinfectant which helps to prevent growth of any microbial contaminants during storage of the buffer. Other optional buffers, solution, and solutions may also be added to the kits as necessary.

Furthermore, kits can optionally include components that are useful in the procedures herein including, but not limited to, capture reagents, developing reagents, reacting surfaces, substrates, means for detection of control samples, instructions and interpretive information explaining how to read results. In preferred embodiments, the kits contain instructions. The instructions preferably contain directions conveying any one or more of the method steps herein.

### EXAMPLES

### Example 1

Preparation of Beta-2 Transferrin Antigen

Antigen beta-2 transferrin was obtained by transforming apo-transferrin to beta-2 transferrin. This transformation involves the usage of neuraminidase, an enzyme that strips away the sialic acid side chains of apo-transferrin. Neuraminidase adsorbed to agarose beads were obtained from Sigma and 200 ul of mixed neuraminidase beads were removed into an eppendorf tube. The beads were washed three times with 10 mM/L sodium acetate. After the third wash, the beads were spun in a microcentrifuge at maximum speed for approximately 10-15 seconds and the supernatant was subsequently removed. Next, 980 ul of Hank's buffered saline solution (HBSS) 1 mg of transferrin (20 ul at 50 ug/ul) were added to the beads. The beads, HBSS and transferrin composition was mixed by inverting repeatedly. The composition was then incubated at 33° C. for approximately 17 hours with continuous mixing. The beads were then spun down [in a microcentrifuge] and the supernatant was removed into a clean eppendorf tube. The protein product was determined to be over 95% pure by mass spectrometry and protein electrophoresis. This product was used as the antigen for the beta-2 transferrin antibody production.

Preparation of Immunized Spleen Cells

Ten BALB/C, 6 week-old female mice were subcutaneously immunized using 100 μl of a 1 mg/ml purified beta-2 transferrin solution mixed with Freund's Complete Adjuvant. The mice were then further immunized 4 times using Freund's Incomplete Adjuvant boosts at three-week intervals by tail vein injection. Periodic serum samples were analyzed for antibody production using ELISA or Western blot. Fourteen days after the final immunization, 10 μg of the antigen was injected into the abdominal cavity of each mouse.

Fusion

Each of the ten immunized mice were euthanized by exsanguination. The spleen of the immunized mice were aseptically extracted and then transferred on a culture dish to which a nylon mesh was fitted. Each spleen was passed through the mesh with a spatula to rid the spleenocytes from connective tissue. The resultant suspension of the spleen cells was collected in a 50 ml centrifugal tube and the cells were peleted by centrifugation (1000 rpm×5 minutes). After peleting, Dulbecco's phosphate buffered saline (DPBS) was used to wash the cells 3 followed by 2 washes using an RPMI 1640 medium. Roughly 1×10⁸ of the mouse spleen cells were mixed with 1×10⁷ of SP210-AG14 myeloma cells. Excess medium was removed by suction and 1 μl of a 50% polyethylene glycol 4000 (trade name; polyethylene glycol the molecular weight of which was 4000, manufactured by Merck and Co., Inc.) solution which was warmed to 37° C. was added to the solution wherein the spleen cells and the myeloma cells were mixed, and the resultant mixture was mixed for 2 minutes.

Next, an RPMI 1640 medium which was warmed to 37° C. was slowly added to the cells and a washing operation was carried out 1 time as described above. After washing, the resultant mixture was allowed to stand in a 5% CO₂ gas incubator at 37° C. for one hour. After the incubation, the cells were washed and suspended in HAT medium. 200 μl of the resultant mixture was added into respective wells of 96-well plate for culturing cells at a density of 1.5×10⁵ cells per well. These plates were cultured in the 5% CO₂ gas incubator for about one week. Viable colonies appeared as early as 3-5 days after fusion. During the culture period, 100 μl of the culture liquid was removed by suction at intervals of about 2-3 days, and then 100 μl of a new HAT medium were added thereto. After the spleen cells and the myeloma cells which were not fused became extinct, the remaining cells were further cultured on a 10% fetal calf serum supplemented RPMI 1640 medium for 1-2 weeks to obtain hybridomas.

Selection of an Anti-Human Beta-2 Transferrin Antibody Forming Hybridoma

Screening was carried out to select the hybridoma generating a desired antibody from the hybridomas obtained in the previous process. As a method for the screening, enzyme immunoassay is used as described herein. As screening objects, the following S substances were selected: human cerebrospinal fluid, purified human apo-transferrin, and purified human beta-2 transferrin. The respective substances were immobilized on plates to prepare plates for enzyme immunoassay which were prepared in the previous step. After reaction for 1 hour and washing, the cultures were reacted with an alkaliphosphatase labeled anti-mouse IgG antibody for 1 hour. After further washing, a color development was performed to clone cells in the wells having an antibody which was not reactive with purified human apo-transferrin and was reactive with purified human beta-2 transferrin and human cerebrospinal fluid.

Establishment of the Hybridoma

The cells in wells wherein the production of the desired antibody was confirmed by an enzyme immunoassay were transferred from the 96-well cell-culturing plates to 24-well cell-culturing plates to increase the number of the cells, and then the cells were cloned 2 times by limiting dilution. Finally, hybridomas were obtained which were able to produce the antibodies (e.g., NP104) that did not specifically bind to human apo-transferrin but that were able to specifically bind to purified human beta-2 transferrin.

Production of a Monoclonal Antibody

For each hybridoma, approximately 0.5 μl of Pristane (2,6,10,14-tetramethylpentadecane, manufactured by Wako Pure Chemicals Industries, Ltd.) was injected into abdominal cavities of BALB/C mice to prepare the mice for ascites fluid production. After about 1 week, the abdominal cavities were inoculated with 5×10⁶ per mouse of the hybridoma cells cultured in vitro. After about 2 weeks, the ascites fluids of the mice were collected and then were primarily purified by the ammonium sulfate salting-out method. The once-purified fluids were again purified on a protein A column to obtain the antibody of the hybridoma having an improved purity.

Cryopreservation of Hybridomas

Cryopreservation of cells assures a back up stock in case clones or antibody production are lost due to contamination, cell death, etc. Cryopreservation also avoids the problems encountered when hybridomas are maintained in culture over long periods of time, such as the need for recloning periodically to avoid changes in antibody characteristics or secretion rates.

Two techniques were used for freezing and recovering cell lines: conventional cryopreservation of cells harvested from culture, and in situ cryopreservation of colonies of new hybridomas and clones growing in 96, or 24-well tissue culture plates (Wells and Price, 1983). The latter technique is invaluable if a fusion results in too many colonies to be evaluated easily or if the screening assay is cumbersome or time consuming. Cryopreservation of primary cell isolations was performed using freezing media containing 10% DMSO.

### Example 2

Confirmation of the Characteristics of the Monoclonal Antibody

To confirm the specificity of the antibody NP104, the antibody was immobilized on plates for enzyme immunoassay and antigen was added to the plates. After reaction for 1 hour, the plates were washed and then a POD labeled transferrin antibody was added thereto. After further reaction for 1 hour, the plates were washed and then color-development of the enzyme was performed.

The monoclonal antibodies of the present invention make it possible to specifically detect whether a body fluid sample contains cerebrospinal fluid. Therefore, it is possible to detect if a cerebrospinal fluid leak is present in a patient with high sensitivity, quickly, efficiently and accurately.

### Example 3

Detection of Beta-2 Transferrin by Western Blotting

The purified beta-2 transferrin obtained in Example 1 is subjected to SDS polyacrylamide electrophoresis (SDS-PAGE) and then blotted on a polyvinylidene difluoride membrane (hereinafter referred to as “PVDF membrane”, Millipore). The PVDF membrane is immersed in PBS supplemented with 1-10% bovine serum albumin (BSA) and left to stand at 4° C. overnight for blocking, followed by thorough washing with PBS containing 0.05% Tween. The PVDF membrane is immersed in the culture supernatant of the hybridoma obtained in Example 2 or a solution of purified antibody obtained in Example 2 at room temperature for 2 hours and washed thoroughly with PBS containing 0.05% Tween. The PVDF membrane is immersed in a solution of an anti-mouse immunoglobulin antibody or anti-rat immunoglobulin antibody as a secondary antibody at room temperature for 1 hour and washed thoroughly with PBS containing 0.05% Tween. The secondary antibody was labeled preliminarily with biotin, an enzyme, a chemiluminescent substance, a radioactive compound or the like. After removing the washing solution completely, a reaction is performed in accordance with the label on the secondary antibody and a check is made for the reactivity with a protein that agrees in the molecular weight to the purified beta-2 transferrin. The results are shown in FIGS. 1-3.

FIG. 1 illustrates binding of commercial transferrin antibody to cerebrospinal fluid (column 2) and to purified human transferrin (column 3). FIG. 2 illustrates binding of beta-2 transferrin antibody to cerebrospinal fluid (column 2) and purified human transferrin (column 3). Column 1 of both figures is a molecular weight marker. As these figures illustrate, the commercial transferrin antibody binds to numerous proteins within the cerebrospinal fluid. See FIG. 1, column 2. On the contrary, beta-2 transferrin antibody generates only one band indicating that it binds specifically to only beta-2 transferrin. See FIG. 2, column 2. Furthermore, while the transferrin antibody binds to transferrin, the beta-2 transferrin antibody does not. See FIG. 2, column 3. This illustrates that beta-2 transferrin not only binds to beta-2 transferrin it also does not bind to transferrin. FIG. 3 illustrates an immunoassay between CSF and beta-2 transferrin in columns 1 and 2 and with transferrin in columns 4 and 5. Beta-2 transferrin antibodies of the present invention bind specifically to beta-2 transferrin in CSF, as is illustrated in columns 1 and 2 while commercially available transferrin antibodies bind to all transferrin isoforms within the CSF.

### Example 4

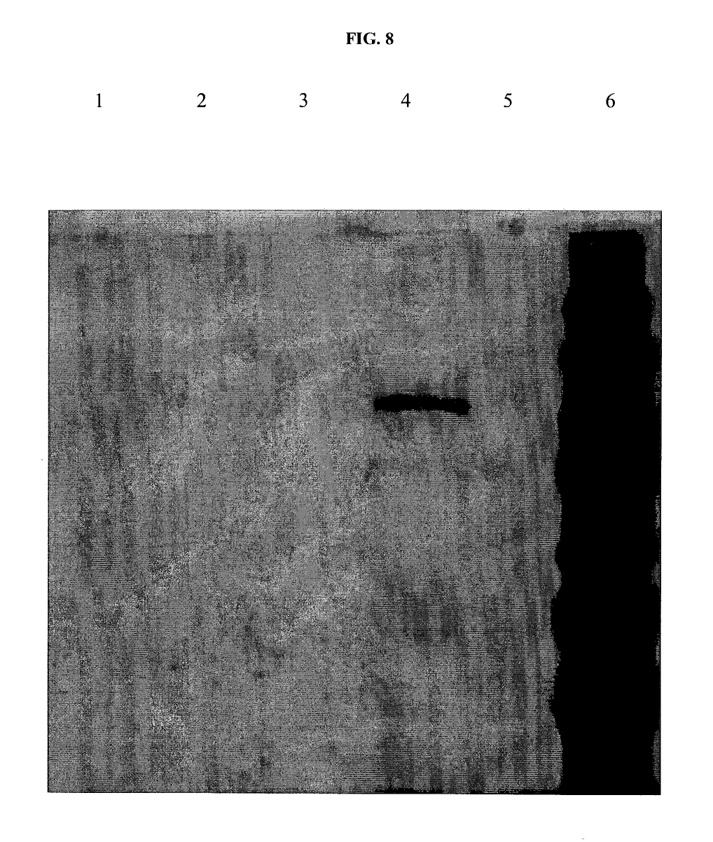

The beta-2 transferrin antibodies of the present invention provide an improved method of detecting cerebrospinal fluid leakage. FIG. 8 illustrates clinical results obtaining using the antibodies herein. Samples of serum or nasal discharge were obtained from four different individuals who were not previously clinically diagnosed with cerebrospinal fluid leakage. The samples were loaded onto an SDS-PAGE gel and then blotted on a PVDF membrane. The PVDF membrane is immersed in PBS supplemented with 1 to 10% BSA and left to stand at 4° C. overnight for blocking, followed by through washing with PBS containing 0.05% Tween. The PVD membrane is then immersed in a monoclonal antibody disclosed herein. Bound beta-2 transferrin antibody was subsequently detected using a secondary antibody such as an anti-mouse antibody. As FIG. 8 illustrates, only column 4 contained beta-2 transferrin that specifically bound to an antibody of the present invention. The individual, whose sample was loaded into column 4 was subsequently diagnosed with cerebrospinal fluid leakage.

What is claimed is:

1. An antibody that specifically binds to human beta-2 transferrin.

2. The antibody of claim 1 wherein the antibody is selected from the group consisting of monoclonal antibodies, humanized antibodies, single chain antibodies and disulfide-stabilized antibodies.

3. The antibody of claim 2 wherein the antibody is a monoclonal antibody.

4. The antibody of claim 1 further comprising a reporter.

5. The antibody of claim 4 wherein the reporter is fluoresce.

6. An apparatus for detecting cerebrospinal fluid leakage said apparatus comprising one or more antibodies that specifically bind to a CSF-specific protein.

7. The apparatus of claim 6 wherein the presence or absences of said CSF-specific protein is indicated by a color change.

8. The apparatus of claim 6 wherein results are obtained in less than 10 minutes.

9. The apparatus of claim 6 further comprising a control region.

10. The apparatus of claim 6 further comprising an application pad for direct contact with a sample.

11. The apparatus of claim 6 wherein the CSF-specific protein is beta-2 transferrin.

12. The apparatus of claim 6 wherein at least of said antibodies is labeled with a reporter.

13. A method for detecting CSF leakage in a patient comprising detection of a CSF-specific protein using a CSF-specific antibody.

14. The method of claim 13 wherein detection is made in a hospital.

15. The method of claim 13 wherein said antibody is bound to a substrate or solid support.

16. The method of claim 13 further comprising a control.

17. The method of claim 13 further comprising the use of a rapid detection device.

18. The method of claim 17 wherein the rapid detection device is a strip test.

19. The method of claim 17 wherein the presence or absence of a CSF-specific protein is determined in less than 10 minutes.

20. The method of claim 13 wherein the CSF-specific antibody is a beta-2 transferrin antibody.

21. The method of claim 20 wherein the beta-2 transferrin antibody is a monoclonal antibody.

22. The method of claim 13 further comprising obtaining a sample from said patient.

23. The method of claim 13 wherein the antibody is labeled.

24. The method of claim 13 wherein said label is colloidal gold.

25. The method of claim 13 wherein the CSF-specific protein is beta-2 transferrin.

26. The method of claim 13 further comprising a second antibody that can specifically bind to the CSF-specific protein.

27. The method of claim 26 wherein said second antibody is bound to a substrate or a solid support.

28. The method of claim of claim 13 further comprising a second antibody that can specifically bind to the CSF-specific protein and CSF-specific antibody complex.

29. The method of claim 26 or 28 wherein binding of the CSF-specific antibody to the second antibody results in a visual change.

30. The method of claim 22, wherein the test sample is selected from a group consisting of: nasal secretions, sinus fluid, aural fluids, serum, tears, saliva, ear discharge and blood.

31. A method for diagnosing CSF leakage in a patient using an antibody that specifically binds a CSF-specific protein.

32. The method of claim 31 wherein the CSF-specific protein is beta-2 transferrin.

33. A method for diagnosing a patient with a condition associated with cerebrospinal fluid leakage comprising the steps of:
providing an apparatus having one or more antibodies that specifically binds a CSF-specific protein wherein the apparatus undergo a change upon exposure to said protein;
contacting a test sample from a patient with said apparatus; and
observing the apparatus for a change.

34. The method of claim 31 wherein the condition is rhinorrhea.

35. The method of claim 31 wherein the condition is otorrhea.

36. The method of claim 31 wherein the condition is congenital perilymphatic fistula.

37. The method of claim 31 wherein the condition is congenital tegmental defects.

38. The method of claim 31 wherein detection is made in a non-hospital setting.

39. The method of claim 31 wherein said antibody is linked to a solid support.

40. The method of claim 39 further comprising a control region.

41. The method of claim 31 wherein the antibody is labeled.

42. The method of claim 41 wherein the label is colloidal gold.

43. A kit for diagnosing cerebrospinal fluid leakage comprising:
a container containing an antibody that specifically binds a CSF-specific protein; and
instructions for use.

44. The kit of claim 43 wherein the CSF-specific protein is beta-2 transferrin.
